(19) United States
(12) Patent Application Publication
(10) Pub. No.: US 2016/0263340 A1
(43) Pub. Date: Sep. 15, 2016

(54) Oxygen Delivery Mask For Transoral Medical Procedures

(71) Applicant: Novlet Marvalyn Fenwick, Milton, DE (US)

(72) Inventor: Novlet Marvalyn Fenwick, Milton, DE (US)

(21) Appl. No.: 14/657,522

(22) Filed: Mar. 13, 2015

(51) Int. Cl. A61M 16/06; A61M 16/10; A61M 16/08

(52) U.S. Cl. CPC A61M 16/0672; A61M 16/0875; A61M 16/1005; A61M 16/0683; A61M 16/0616; A61M 16/0816; A61M 2202/0208

(57) ABSTRACT

An oxygen delivery mask for transoral medical procedures that provides an efficient means for safely delivering oxygen to patients at higher flow rates. The oxygen delivery mask comprises a mask body defining a wearable face covering that includes a nasal compartment, a mouth opening, two fastening apertures, four mask inlets and a mask outlet with an integral delivery tube that includes four inlet ports and a sealed hole and an exhaust port. The oxygen delivery mask is shaped to contour to the face of a wearer, and when in use, enables input gas to be directed from the delivery tube into the inside of the mask and output exhaust gas to be expelled from the inside of the mask. The oxygen delivery mask thereby allows for treatments with oxygen flow rates greater than three liters/minute to be delivered to patients without concern of causing trauma to the patient's nostrils.

### BRIEF DESCRIPTION OF THE DRAWINGS

FIG. 1 is a front elevational view of an oxygen delivery mask built in accordance with the present invention.

FIG. 2 is a front elevational view of the mask body and exhaust port of an oxygen delivery mask built in accordance with the present invention.

FIG. 3 is a partial back perspective view of the delivery tube of an oxygen delivery mask built in accordance with the present invention.

FIG. 4 is a side elevational view of an oxygen delivery mask built in accordance with the present invention in place on a user.

### DETAILED DESCRIPTION OF THE INVENTION

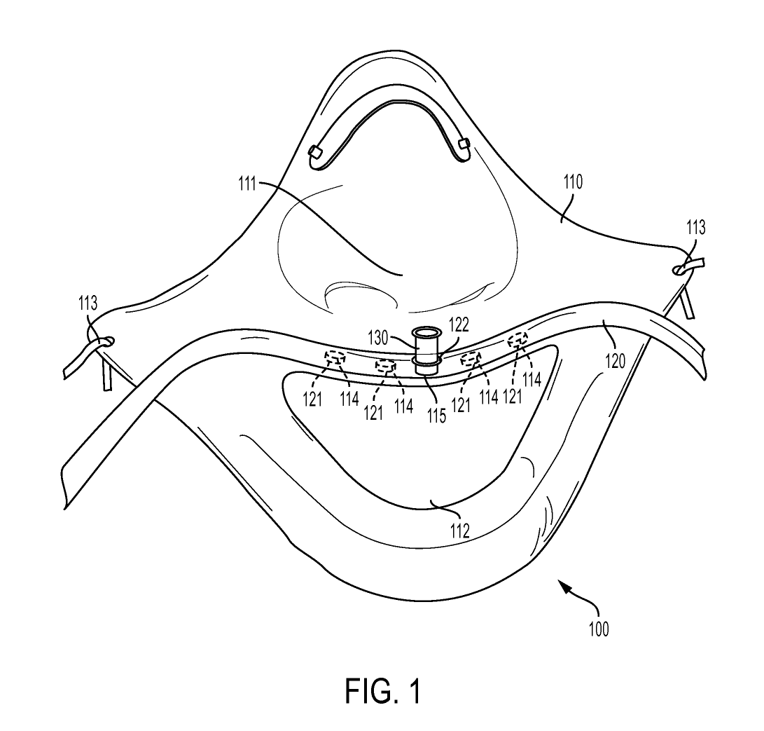

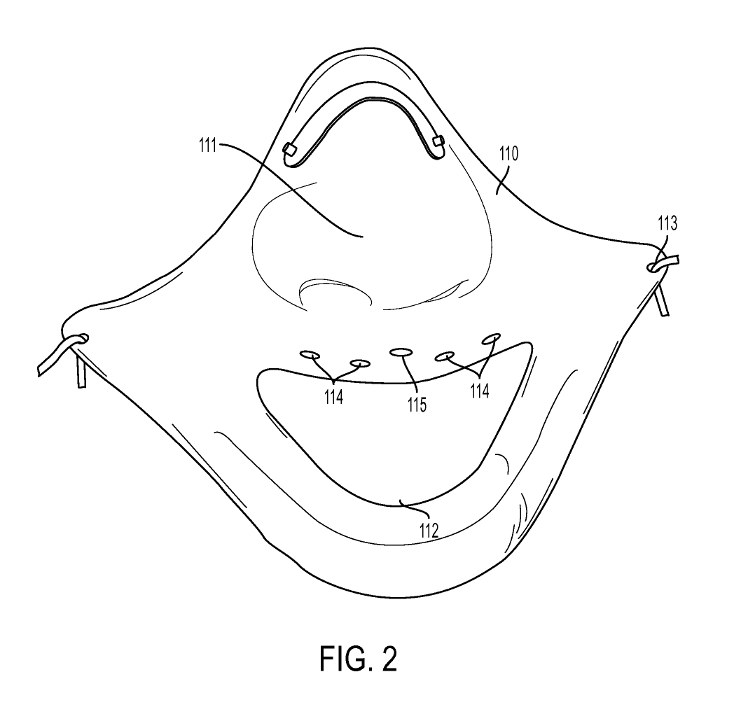

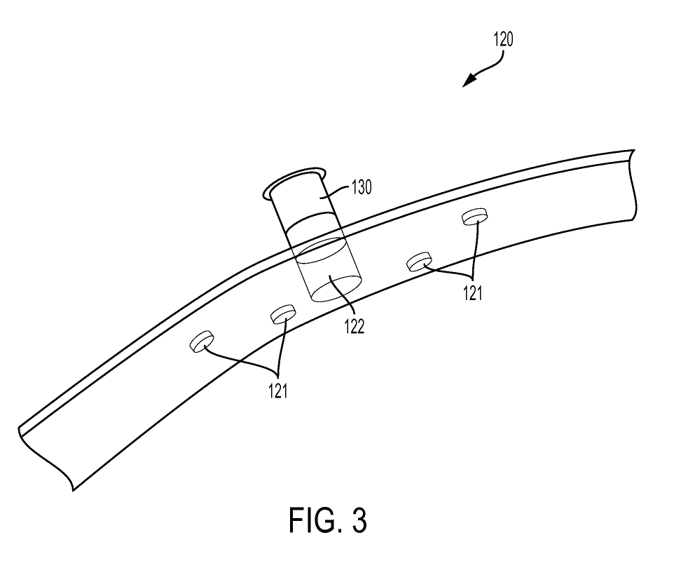

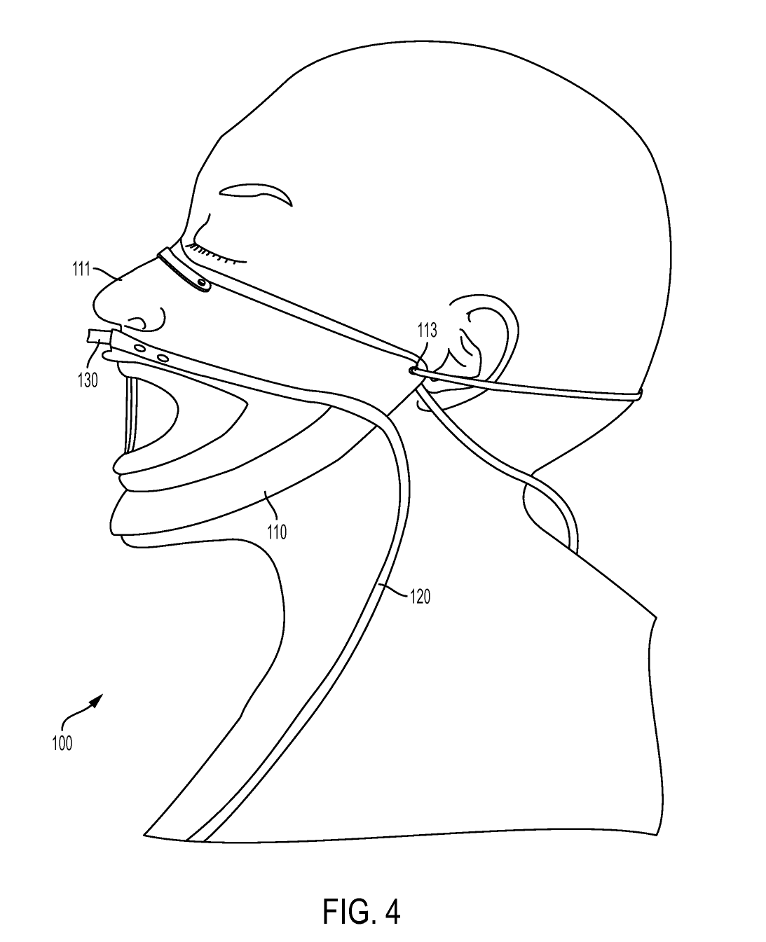

Referring now to the drawings and in particular FIGS. 1, 2, 3, and 4, an oxygen delivery mask 100 built in accordance with the present invention includes a mask body 110 and a delivery tube 120. The mask body 110 defines a wearable face covering that includes a nasal compartment 111, a mouth opening 112, two fastening apertures 113, four mask inlets 114 and a mask outlet 115. The nasal compartment 111 of the mask body 110 is shaped to mirror the shape of a human nose so as to encase a wearer's nose without deforming it and the mouth opening 112 defines an aperture in the mask body 110 disposed underneath the nasal compartment 111. Accordingly, the mask body 110 fully covers the nose of a wearer and forms a seal forming portion that contours the wearer's face with an opening around the mouth. The fastening apertures 113 are disposed on opposing sides of the mask body 110 and enable the mask body 110 to receive securing structures, such as conventional securing strings, straps, or ties.

The mask inlets 114 and the mask outlet 115 each define a discrete aperture in the mask body 110 that allows gas to be directed through from the side of the mask body 110 adjacent to a wearer's face (the “inside” of the mask)) to the side of the mask body 110 opposite to a wearer's face (the “outside” of the mask) or, in the alternative, from the outside of the mask body 110 to the inside of the mask body 110.

The delivery tube 120 defines a conventional air delivery tube that additionally includes four inlet ports 121 and a sealed hole 122. The inlet ports 121 each define a tube portion that branches off from the delivery tube 120, protruding its surface. Accordingly, the inlet ports enable the dispensing of gas that is traveling through the delivery tube 120.

An exhaust port 130 defines a one way valve which is placed over the mask outlet 115 so as to enable gas inside the mask body 110 to be vented to the outside of the mask body 110. The sealed hole 122 allows for the placement of the discrete exhaust port 130 through a central area in the delivery tube 120 without enabling gas flowing through the delivery tube 120 to escape or otherwise mixing of gas in the delivery tube 120 with gas from inside the mask body 110.

The oxygen delivery mask 100 is defined by the mask body 110 having the delivery tube 120 in place on the mask body 110 such that each of the inlet ports 121 is aligned with and engages a corresponding mask inlet 114 and the sealed hole 122 is aligned with the mask outlet 115 so that the exhaust port 130 that extends therefrom can extend beyond the delivery tube 120. Protruding from the surface of the delivery tube 120, the inlet ports 121 each extend into their aligned mask inlet 114 so as to allow gas flowing through the delivery tube 120 to be directed into the inside of the mask body 110. Accordingly, it is contemplated that that the inlet ports 121 and mask inlets 114 are positioned to provide an optimum delivery of input gas (such as oxygen) to the area inside the mask under the nasal compartment 111 and above the mouth opening 112.

The exhaust port 130 is seated in the mask outlet 115 such that it forms an outlet channel that allows the flow of gas from the inside of the mask to the outside of the mask. As such, the exhaust port 130 allows exhaust gas (such as CO2) to be vented from the inside of the mask. The sealed hole 122 allows the exhaust port 130 to extend directly from the mask body 110 beyond the delivery tube 120.

It is appreciated that because the exhaust port 130 is constructed to extend beyond the delivery tube 120 away from the mask body 110, the end of the exhaust port 130 distal to the mask body 110 may be structured to accommodate a conventional Leur-Lock end of a CO2 sampling tube to enable CO2 monitoring.

In use, the oxygen delivery mask 100 enables the delivery of oxygen without the need for a conventional nasal cannula or other nasal insert based structures. Advantageously, the use of the oxygen delivery mask 100 allows for oxygen flow rates greater than three (3) liters/minute to be delivered to patients more comfortably by eliminating common problems nasal insert use, such as burning sensations in the nostrils and nose bleeds, while still minimizing the risk of low oxygen saturation and inadequate oxygen delivery.

In the preferred embodiment, the mask body 110 is constructed of a transparent material.

The present invention has been shown and described herein in what is considered to be the most practical and preferred embodiment. It is recognized, however, that departures may be made therefrom within the scope of the invention and that obvious modifications will occur to a person skilled in the art.

What is claimed is:

1. An oxygen delivery mask for transoral medical procedures, comprising:
a mask body defining a wearable face covering having an inside relative to the face of wearer and an outside relative to the face of wearer, wherein the mask body includes a nasal compartment, at least one mask inlet and at least one mask outlet;
at least one delivery tube defining an air delivery tube that includes at least one inlet port, wherein said delivery tube is integral with the mask body such that each of the at least one inlet ports engage one mask inlet so as to enable gas flowing through the delivery tube to be directed to the inside of the mask body; and
an exhaust port integral with the mask outlet, wherein said exhaust port is configured to allow gas inside the mask body to flow to the outside of the mask.

2. The oxygen delivery mask of claim 1, wherein said at least one delivery tube additionally includes a sealed hole.

3. The oxygen delivery mask of claim 2, wherein said exhaust port defines a channel that extends from the mask body and passes through the sealed hole.

4. The oxygen delivery mask of claim 1, wherein said exhaust port defines a channel that extends from the mask body.

5. The oxygen delivery mask of claim 1, wherein the mask body is shaped to contour to the face of a wearer, with the nasal compartment covering the nose of a wearer to form a sealed area inside the mask under the nasal compartment.

6. The oxygen delivery mask of claim 1, wherein the mask body additionally includes a mouth opening.

7. The oxygen delivery mask of claim 1, wherein:
the mask body includes four mask inlets; and
the delivery tube includes four inlet ports.

8. The oxygen delivery mask of claim 1, wherein the mask body additionally includes two fastening apertures.

9. The oxygen delivery mask of claim 1, wherein each of the at least one mask inlets defines a discrete aperture in the mask body positioned under the nasal compartment.

10. The oxygen delivery mask of claim 9, wherein:
each of the at least one inlet ports defines a tube portion that branches off from the delivery tube, protruding from the surface of the delivery tube; and
each of said at least one inlet ports engage their corresponding mask inlet by extending into the corresponding mask inlet.

11. An oxygen delivery mask for transoral medical procedures, comprising:
a mask body defining a wearable face covering having an inside relative to the face of wearer and an outside relative to the face of wearer, wherein the mask body includes a nasal compartment and at least one mask outlet;
at least one delivery tube defining an air delivery tube, wherein said delivery tube is integral with the mask body and configured to enable gas flowing through the delivery tube to be directed to the inside of the mask body; and
an exhaust port integral with the mask outlet, wherein said exhaust port is configured to allow gas inside the mask body to flow to the outside of the mask.

12. The oxygen delivery mask of claim 11, wherein said at least one delivery tube additionally includes a sealed hole.

13. The oxygen delivery mask of claim 12, wherein said exhaust port defines a channel that extends from the mask body and passes through the sealed hole.

14. The oxygen delivery mask of claim 11, wherein the mask body additionally includes two fastening apertures.

15. The oxygen delivery mask of claim 11, wherein the mask body is shaped to contour to the face of a wearer, with the nasal compartment covering the nose of a wearer to form a sealed area inside the mask under the nasal compartment.

16. The oxygen delivery mask of claim 15, wherein the mask body additionally includes a mouth opening.

17. The oxygen delivery mask of claim 16, wherein:
the mask body includes at least one mask inlet
the delivery tube includes at least one inlet port; and
said delivery tube is configured to enable gas flowing through the delivery tube to be directed to the inside of the mask body through each of the at least one inlet ports engaging one mask inlet.

18. The oxygen delivery mask of claim 17, wherein:
the mask body includes four mask inlets; and
the delivery tube includes four inlet ports.

19. The oxygen delivery mask of claim 17, wherein each of the at least one mask inlets defines a discrete aperture in the mask body positioned under the nasal compartment.

20. The oxygen delivery mask of claim 19, wherein:
each of the at least one inlet ports defines a tube portion that branches off from the delivery tube, protruding from the surface of the delivery tube; and
each of said at least one inlet ports engage their corresponding mask inlet by extending into the corresponding mask inlet.
